(19) United States
(12) Patent Application Publication
(10) Pub. No.: US 2004/0033232 A1
(43) Pub. Date: Feb. 19, 2004

(54) Methods and compositions for in vivo clearance of pathogens

(76) Inventors: Elliot R. Ramberg, Hollywood, FL (US); Martin J. Lopez, Sunrise, FL (US)

Correspondence Address: Buchanan Ingersoll, P.C., One Oxford Centre, 20th Floor, 301 Grant Street, Pittsburgh, PA, 15219, US

(21) Appl. No.: 10/459,771

(22) Filed: Jun. 12, 2003

Related U.S. Application Data
(60) Provisional application No. 60/388,238, filed on Jun. 13, 2002.

(51) Int. Cl. A61K 39/395; C12N 5/08

(52) U.S. Cl. 424/178.1; 435/372

(57) ABSTRACT

The present invention comprises methods and compositions using biological factors, such as complement components, and manipulation of cells of erythroblastic lineage and myeloid lineage to facilitate clearance of pathologic targets from the blood stream in specific phagocytic compartment.

### BENEFIT OF PRIOR PROVISIONAL APPLICATION

This utility patent application claims the benefit of co-pending U.S. Provisional Patent Application Serial No. 60/388,238, filed Jun. 13, 2002, entitled “Methods and Compositions For In Vivo Clearance Of pathogens” having the same named applicants as inventors, namely, Elliot R. Ramberg and Martin J. Lopez. The entire contents of U.S. Provisional Patent Application Serial No. 60/388,238 is incorporated by reference into this utility patent application.

### BACKGROUND OF THE INVENTION

1. Field of the Invention

This invention relates to the field of immunology. In particular, it is directed to methods and compositions for the in-vivo clearance of pathologic and other targets from the peripheral blood of a patient. The methods comprise administering to the patient at least one sensitized erythrocyte having a molecule pair antibody that is capable of binding a pathological agent at a site other than the CR1 receptor. The methods of the present invention for clearing blood-borne pathogens in a patient also include administering an effective amount of a molecule pair, allowing the molecule pair to bind to a specific site on at least one erythrocyte ghost surface for forming a sensitized erythrocyte ghost molecule pair, and allowing the erythrocyte ghost molecule pair to bind to a specific pathological target in the patient's blood to any site on the erythrocyte resulting in an erythrocyte ghost molecule pair pathological target, and clearing the erythrocyte ghost molecule pair pathological target from the patient's blood.

2. Description of the Background Art

The present invention concerns methods and compositions for the in-vivo clearance of pathologic and other targets from the peripheral blood. These targets may include the following but are not limited to microbial organisms such as virus, bacteria, rickettsia and fungi, agents of biological and chemical warfare, dysplastic and metastatic cancer cells, autoimmune antibodies and any molecule mediating a pathologic or other process, or present in the body. Appropriate targets are those that can be bound by a binding partner to form complexes such as immune complexes (IC) that can then be removed from the circulation through natural processes such as phagocytosis. In particular, the invention comprises methods and compositions using biological factors, such as antibodies and complement components, and manipulation of cells of erythroblastic lineage and myeloid lineage to facilitate clearance of the pathologic targets from the blood stream in multiple phagocytic compartments by different natural clearance mechanisms.

Recognition of non-self is a fundamental trait for assuring survival in all forms of living organisms. During evolution two general systems of immunity have emerged: innate or natural immunity, and adaptive (acquired) or specific immunity. Innate immunity in mammals appears to play an important role in the early phase of defense and also stimulates the clonal response of adaptive immunity.

In general, the immune defense system is comprised of two parts, the humoral immune system, and the cellular immune system. Humoral immune responses are mediated by antibodies, natural glycoproteins secreted by B-cells in response to specific antigens such as proteins from pathogens or expressed on normal tissues.

Cell-mediated immune responses result from the interactions of cells, including antigen presenting cells, B lymphocytes (B cells), and T lymphocytes (T cells). The cellular immune system is comprised of cells of myeloid lineage, the polymorphonuclear granulocytes including neutrophils, basophiles, and eosinophils; the circulating monocytes (minimally phagocytic), and the fixed tissue monocytes including the mature Kupffer cells in the liver, the cells of the intraglomerular mesangium of the kidney, the alveolar macrophages in the lung, the serosal macrophages, the brain microglia, spleen sinus macrophages and lymph node sinus macrophages. These phagocytic cells are characterized in Table III in terms of their surface receptors and their granular contents.

The immune response is initiated by the recognition of foreign antigens by various kinds of cells, principally macrophages or other antigen presenting cells leading to activation of lymphocytes that specifically recognize a particular foreign antigen resulting in its elimination. Elimination of a foreign antigen involves complex interactions that lead to helper functions, stimulator functions, and suppressor functions among others. The power of the immune system's responses must be carefully controlled at multiple sites, for stimulation and suppression, or the response will either not occur, be over responded to or not continue after pathologic target elimination.

The recognition phase of response to foreign antigens consists of the binding of foreign antigens to specific receptors on immune cells. These receptors generally exist prior to antigen exposure. Recognition can also include interaction with the antigen by macrophage-like cells or by recognition by factors within serum or bodily fluids.

In the activation phase, lymphocytes undergo at least two major changes. They proliferate, leading to expansion of the clones of antigen-specific lymphocytes and amplification of the response, and the progeny of antigen-stimulated lymphocytes differentiate either into effector cells or into memory cells that survive, ready to respond to re-exposure to the antigen. There are numerous amplification mechanisms that enhance this response.

In the effector phase, activated lymphocytes perform the functions that may lead to elimination of the antigen and establishment of the immune response. Such functions include cellular responses, such as regulatory, helper, stimulator, suppressor or memory functions. Many effector functions require the combined participation of cells and cellular factors. For instance, antibodies bind to foreign antigens and enhance their phagocytosis by blood neutrophils and mononuclear phagocytes, free and fixed.

In general, the humoral immune system function results in the production of antibody specific to an invading immunogenic target and is mediated by T lymphocyte processing of the immunogen and transferring or presenting it to the B lymphocytes to initiate antibody production specific for the immunogen. All of the mature monocytes, due to their increase in size post migration into specific tissues, remain fixed and cannot themselves reenter the circulatory system. These mature monocytes phagocytize a microbial invader or other immunogenic target in the form of an opsonized immune complex (IC) followed by clearance of the IC from the body. Thus, the cellular immune defense in vertebrates has evolved to include antigen processing, antibody producing cells (lymphocytes), and macrophages of two distinct myeloid lineages. The resultant function of both systems is the clearance of any foreign target from the body.

### SUMMARY OF THE INVENTION

A method for blood-borne pathogen clearance in a patient in vivo is described. The method of the present invention comprises preparing at least one erythrocyte ghost having at least one senescence marker, sensitizing at least one of the erythrocyte ghosts with at least one molecule pair ex vivo to form a sensitized erythrocyte ghost molecule pair, administering an effective amount of the sensitized erythrocyte ghost molecule pair to a patient, and effecting the binding of the sensitized erythrocyte ghost molecule pair to a specific pathological agent present in the patient's blood resulting in an erythrocyte ghost molecule pair pathological agent, and clearing the erythrocyte ghost molecule pair pathological agent from the patient's blood.

Another embodiment of this invention provides a method for forming a sensitized erythrocyte. This method comprises obtaining at least one erythrocyte, biotinylating the erythrocyte to form a biotinylated erythrocyte, obtaining at least one monoclonal antibody specific to a target, biotinylating the monoclonal antibody to form a biotinylated monoclonal antibody, binding the biotinylated erythrocyte to avidin, and binding the avidin having the biotinylated erythrocyte to the biotinylated monoclonal antibody to form a sensitized erythrocyte.

Another embodiment of this invention provides a method for forming a sensitized erythrocyte comprising obtaining at least one erythrocyte, biotinylating the erythrocyte to form a biotinylated erythrocyte, obtaining at least one monoclonal antibody specific to a target, biotinylating the monoclonal antibody to form a biotinylated monoclonal antibody, binding the biotinylated erythrocyte to streptavidin, and binding the streptavidin having the biotinylated erythrocyte to the biotinylated monoclonal antibody to form a sensitized erythrocyte.

Another embodiment of this invention provides a method for forming a sensitized erythrocyte comprising obtaining at least one erythrocyte, selecting a highaffinity binding pair, treating the erythrocyte with a first member of the high-affinity binding pair, obtaining at least one monoclonal antibody specific to a target, treating the monoclonal antibody with a second member of the high-affinity binding pair, and combining the treated erythrocyte with the treated monoclonal antibody to form a sensitized erythrocyte.

This invention provides a composition comprising an erythrocyte and a molecule pair antibody wherein the molecule pair antibody is bound to the erythrocyte at the Rho (D) locus of the erythrocyte, and wherein the molecule pair antibody comprises IgG anti Rho (D) covalently bound to a monoclonal antibody specific for a target, and wherein the IgG anti Rho (D) has an Fc region.

In another embodiment of this invention, a method is provided for prolonging the ability to eliminate pathological agents from the blood of a patient comprising administering to a patient at least one sensitized erythrocyte ghost having a molecule pair antibody complex that is capable of binding a pathological agent, including wherein the sensitized erythrocyte ghost includes a band 3 surface polypeptide, and including wherein the sensitized erythrocyte ghost exhibits no surface appearance of phosphatidylserine, and administering an effective amount of an anti-malaria drug to the patient to prevent elimination of the sensitized erythrocyte ghost molecule pair antibody for prolonging the patient's ability to eliminate the pathological agent.

In yet another embodiment of this invention, a method for elimination of pathological agents from the blood of a patient is provided. This method comprises administering to the patient at least one sensitized erythrocyte having a molecule pair antibody that is capable of binding a pathological agent at a site other than the CR1 receptor of the sensitized erythrocyte and eliminating the pathological agent from the patient's blood, and including adding an effective amount of soluble Fc for inhibiting the clearance reaction of the sensitized erythrocyte molecule pair.

Another embodiment of this invention provides a method for blood-borne pathogen clearance in a patient in vivo comprising administering to a patient an effective amount of a molecule pair, wherein the molecule pair is prepared using humanized or non-humanized antibodies, allowing the molecule pair to bind to a specific site on at least one erythrocyte surface different from CR1 thereby forming a sensitized erythrocyte molecule pair and allowing the sensitized erythrocyte molecule pair to bind to a specific pathological target in the patient's blood to any site on the erythrocyte other than the CR1 resulting in an erythrocyte molecule pair pathological target, and clearing the erythrocyte molecule pair pathological target from the patient's blood.

Further, another embodiment of the present invention provides a method for blood-borne pathogen clearance in a patient in vivo comprising administering to a patient an effective amount of a molecule pair, wherein the molecule pair is prepared using humanized or non-humanized antibodies, allowing the molecule pair to bind to a specific site on at least one erythrocyte ghost surface thereby forming a sensitized erythrocyte ghost molecule pair, and allowing the sensitized erythrocyte ghost molecule pair to bind to a specific pathological target in the patient's blood to any site on the erythrocyte resulting in an erythrocyte ghost molecule pair pathological target, and clearing the erythrocyte ghost molecule pair pathological target from the patient's blood.

In yet another embodiment, a method for elimination of pathological agents from the blood of a patient is provided comprising administering to the patient at least one sensitized erythrocyte having a molecule pair antibody that is capable of binding a pathological agent at a site other than the CR1 receptor including wherein the molecule pair antibody comprises two antibodies that are covalently linked, wherein one of the antibodies is specific for binding to an erythrocyte receptor site and the other antibody is specific to the pathological agent, and including wherein the antibody specific to the pathological agent possesses an intact Fc region, and eliminating the pathological agent from the patient's blood independent of the CR1 exchange reaction.

Another embodiment of this invention provides a method for elimination of pathological agents from the blood of a patient comprising administering to the patient at least one sensitized erythrocyte having a molecule pair antibody that is capable of binding a pathological agent at a site other than the CR1 receptor, eliminating the pathological agent from the patient's blood independent of the CR1 exchange reaction and repeating the above steps for extending the ability to eliminate pathological agents from the blood of the patient.

In yet another embodiment of this invention, a method for blood-borne pathogen clearance in a patient in vivo is provided comprising preparing at least one erythrocyte ghost having at least one senescence marker, sensitizing at least one of the erythrocyte ghosts with at least one molecule pair ex vivo administering an effective amount of the sensitized erythrocyte ghost molecule pair to a patient, and allowing the sensitized erythrocyte ghost molecule pair to bind to a specific pathological agent present in the patient's blood resulting in an erythrocyte ghost molecule pair pathological agent, and clearing the erythrocyte ghost molecule pair pathological agent from the patient's body.

### BRIEF DESCRIPTION OF THE TABLES

Table I depicts the clearance of immune complexes (IC) by direct and indirect methods. The direct methods involve the attachment of the opsonized (C3b bound) IC to phagocytic cells and its clearance. The indirect methods involve the attachment of the target to an antibody pair sensitized erythrocyte (E) (intact E or ghost E) with its subsequent clearance from the circulation.

Table II depicts a process comparison between heteropolymer (HP) CR1 exchange reaction IC clearance and molecular pair (MP) selective target elimination (STE) IC clearance with its four embodiments.

Table III details the surface receptors expressed in all the phagocytic cell compartments and their granular content.

Table IV lists the additional sites for possible attachment of the MP to the E surface.

Table V is a glossary that sets forth the meaning of terms used in this utility patent application.

### DETAILED DESCRIPTION OF THE INVENTION

Formation of the IC (immune complex) is a normal part of the immune process. Immune complexes are present in the circulation of healthy individuals, and it is only under some pathological conditions that significant amounts of IC trigger the sequence of injurious events that lead to disease. Most of the ICs in the circulating blood are rapidly cleared by the phagocyte system. The efficiency of antigen elimination from the circulation by the phagocytic cells depends on factors such as affinity of the interaction between the antigen and the antibody molecule; ratio of antigen to antibody and concentration of both type of molecules; and the modification of IC after its formation and/or deposition. Concerning the latter, ICs activate the complement system through both the classical and alternative pathways as known by those persons skilled in the art, although evidence in human beings indicates that the classical pathway is principally involved. IC deposition in tissues may lead to hypersensitivity, with subsequent complement activation causing an inflammatory response. This type of hypersensitivity is typically manifested as serum sickness, glomerulonephritis, rheumatoid arthritis and systemic lupus erythematosus.

The Complement System in higher vertebrates plays an important role as an effector of both innate and the acquired immune response. This system is composed of a series of plasma proteins involved in the immune response to invading pathologic targets. The complement system generates a membrane attack complex (MAC) that promotes the direct lysis of microorganisms in the circulation. From a biological standpoint it is probable that ICs with the greatest pathological potential are primarily those that can activate plasma mediator systems such as the complement system. However, to avoid inadvertent complement-mediated autologous tissue damage, host cells, particularly those that have close contact with plasma such as erythrocytes and endothelial cells, express a number of fluid-phase and membrane-bound inhibitors of complement activation. Human erythrocytes for example contains a glycosylphosphatidylinositol (GPI)-anchored membrane regulator of complement called decay-accelerating factor (DAF) which inhibits the C3 convertase activity) of both the classical and alternative pathways.

In general pathologic targets opsonized with antibody possessing an intact Fc region and C3b as a result of complement fixation of the IC, are more efficiently phagocytized by both the circulating polymorphonuclear granulocytes (PMNs), and the hepatic and splenic macrophages. This effect is mediated by Fcγ receptors and C3b (or CR1) receptors on the phagocytic cell surface. Tables I: A and I: B represent this direct target clearance.

Another process for in vivo clearance of pathologic targets, represented in Table I: C, involves indirect clearance of the complement opsonized IC by attachment to the primate erythrocyte (E) CR1 surface receptors (E CR1). The reaction is rapid and the IC/C3b complex attached to E CR1 is rapidly shunted to the liver and spleen for phagocytosis via the erythrocyte-immune-complex (E-IC) clearance reaction by the fixed tissue monocytes.

Based on this indirect in vivo clearance mechanism Ronald Taylor presented a strategy wherein a heteropolymer (HP), always defined as IgG anti target-IgG anti CR1, is attached to the primate E forming E HP. The sensitized E HP will rapidly bind the specific target in the circulatory system and attached to the “privileged” CR1 site. Once bound to the erythrocyte and carried to the liver, the CR1-HP-target immune complexes should be recognized, stripped from E, phagocytosed, and destroyed by macrophages in the liver (an to a lesser extent spleen) with subsequent recycling of CR1 deficient E. This indirect target clearance mechanism has some drawbacks that will be later discussed, while still demonstrating fast and somewhat efficient in vivo target clearance in the circulation.

Immune complex clearance in the presence of activated complement component C3b leads to a more efficient clearance mechanism based upon the presence of CR1 receptors on phagocytic cells and on the primate E. However, the presence of CR1 receptors on primate red blood cells competitively inhibits the PMN uptake and for the most part directs the IC-C3b complex to the fixed monocytes in the liver and spleen for clearance by the CR1 transfer reaction, J. Immunol., Vol. 145, pages 4198-4206 (1990) In the present invention a different indirect in vivo target clearance process was designed and it is called Selective Target Elimination (STE). STE involves a number of embodiments that in general can be used for clearance of pathologic or other targets from the peripheral blood. These embodiments, STE I and STE II, are intended to provide a better target clearance system than those currently available.

In STE I (Table I: E), a molecule pair (MP) always defined as IgG anti target-Fab anti any immunogenic site on the E surface other than CR1, is attached to the primate E forming E MP. Post MP injection, the sensitized E MP will rapidly bind the specific target in the circulation to any site on E other than the CR1 site resulting in phagocytosis of the E MP/target/C3b opsonized complex primarily in hepatic and splenic monocytes, and possibly including the circulating PMNs. The potential advantages and downsides of STE I is discussed herein.

STE II embodiments were designed to improve E MP target clearance, wherein the MP ex vivo sensitizes erythrocyte ghosts (Egs). Post-transfusion into the body the Eg MP binds targets present in the circulation, and directs the pathologic target to the privileged apoptotic or scenescent cell natural clearance system, utilized to clear trillions of apoptotic cells daily. STE II would provide a short passive immunity period (STE IIa, Table I: F) or a prolonged period of passive immunity (STE IIb, Table I: G)

All of the aforementioned processes will support efficient and rapid in vivo target clearance by activation of a naturally occurring process. E HP functions by utilization of the “privileged” CR1 transfer reaction. E MP STE I functions by utilization of the phagocytic cell surface receptors (PMNs and macrophages). Eg MP (STE IIa, STE IIb, and STE IIc) function by the use of the natural apoptotic cell clearance mechanism in the bloodstream.

The E HP and E MP/Eg MP processes will now be presented and will be characterized in terms of their overall benefits, downsides, and period of immunity conferred.

### Methodologies for RBC Sensitation in STE

The attachment of any antibody specific for a pathologic target to a red blood cell for in vivo pathologic target clearance can be performed by any number of strategies that will in vivo or ex vivo sensitize the RBCs or the RBC membranes. These strategies include the use of antibody pairs namely the molecule pair that attaches the target specific monoclonal antibody to any surface immunogenic site on the RBC surface or RBC membrane surface, and the heteropolymer, also an antibody pair that attaches the target specific mAb only to the CR1 receptor on the RBC surface or the RBC membrane surface. These aforementioned strategies are presented within this document.

Other strategies to sensitize the RBC cell membrane surface to the target specific monoclonal antibody may also include use of the binding pair avidin and biotin. Any high affinity binding pair may also be employed.

It has been demonstrated that randomly biotinylated RBCs generated by use of biotin N-hydroxysuccinimide ester (BNHS) followed by streptavidin treatment can result in the binding of 50,000 molecules of biotinylated IgG (target specific) to the RBC surface. This strategy includes the direct avidin attachment to biotinylated membrane proteins; lipids, and sugars; and the subsequent attachment of b-Ab to avidin exposed biotinylated RBCs. Streptavidin also mediates attachment of b-Ab (target specific) to biotinylated ligands such as lectin or antibody which can be specifically bound to an RBC membrane receptor post avidin exposure to the biotinylated RBS surface.

Additional methods also include the use of RBC cholesterol and other surface component exchange reactions resulting in biotinylation of the RBC surface followed by avidin exposure and subsequent binding of b-mAb specific for the target.

Similar RBC sensitization was achieved by use of biotin-phosphatidylethanolamine (biotin-PE). Herein, in this exchange reaction preincubation of RBCs in a aqueous dispersion of biotin-PE provides for binding of 500,000 avidin molecules per cell that can be used to attach a target specific monoclonal antibody.

These or any other methods resulting in the sensitization of RBCs to a target specific mAB are included as embodiments of the present invention.

### Selection of the Appropriate RBC Sensitization Process

As discussed in the embodiments of STE I and STE II presented herein, concern must be placed on the ability of the RBC sensitization process to itself fix complement. Use of any RBC sensitization process must take into consideration the ability of the sensitization to trigger complement fixation. In general long term protective STE embodiments require no complement fixation by the RBC sensitization process, and possesses a complement trigger post complexation of the pathologic target with the sensitized RBC. On the other hand short term protective STE embodiments are unaffected by complement fixation at the stage of RBCs sensitization.

Therefore, the RBC sensitization process chosen for STE embodiments may or may not be dependent on their ability to fix complement.

### Phagocytosis: The Programming of Indirect In Vivo Target Clearance

The binding of the opsonized immune complex to erythrocytes (E) can lead to uptake and destruction of the erythrocyte-immune complex by phagocytosis. It is also known that the pathway and compartment selected for processing the erythrocyteimmune complex is dependent upon the number of immune complexes bound per erythrocyte and the homogenous surface distribution of available surface binding sites.

Once the target binds the primate E CR1 site, either directly or indirectly, it is cleared solely by passage primarily through the liver and secondarily through the spleen. In this scenario the circulating granulocyte phagocytic cell is excluded from the phagocytic clearance of the immune complex. It is known by those skilled in the art that the factor controlling compartmentalization of phagocytosis is the manner with which the immune complex interacts with the E. If the immune complex is attached to the CR1 site on E, it is precluded from granulocyte phagocytosis, known by those skilled in the art to be a result of the disperse patches of CR1 clusters on the E surface. The polymorphonuclear granulocytes for phagocytosis of the IC must recognize the even placement of the IC on the E generated by a homogeneous distribution of IC binding sites on the entire E surface; not provided by the CR1 discrete disperse patches.

It is the object of the present invention that attachment of the IC at a site other than CR1 on the E will allow the E IC complexes not only to be phagocytized in the liver and spleen, but possibly also in the circulating PMN phagocytic compartment, thereby increasing the kinetics and overall efficiency of in vivo target clearance beyond that provided by the CR1 exchange reaction exclusively.

### E HP: Use of the “Privileged” CR1 Site on the Primate E for Rapid in Vivo Target Clearance in the Circulation Via the CR1 Transfer Reaction (Table I: D and Table II)

A heteropolymer is defined as a polymer comprised of two antibodies of differing specificity, one always being the IgG anti-CR1 antibody and the other being the IgG anti-pathologic target. The heteropolymer is used as a surrogate to replace C3b opsonization of the immune complex by directly attaching the immune complex to the E CR1 site via the IgG anti-CR1 of the HP. The following sequence of events will briefly describe the E HP clearance of a pathogen:

1. E is sensitized, preferably in vivo with a two-specificity antibody pair, HP, such as one described above.

2. E HP interacts by binding the pathologic microbe, and no complement is required to be fixed or activated.

3. The E HP/target complex will travel to the liver and spleen in the normal circulation.

4. The CR1-HP-target grouping is stripped from E by the liver macrophages through a mechanism of clearance called the Transfer Reaction, J. Immunol., Vol. 145, Pages 4198-4206 (1990). This reaction involves proteolysis of the E CR1/HP target complex.

5. The E HP/Target complex, and E HP sans target will both undergo the transfer reaction resulting in HP and HP/target phagocytosis with the removal of the erythrocyte CR1 receptor.

6. The E is released to the circulatory system deficient in CR1 surface receptors.

### Dynamics of the HP CR1 Transfer Reaction

Binding of the EHP/target complex to the CR1 site on the primate E initiates target movement to the liver and spleen. The E HP or E HP target complex, both sans complement bind to the FcγR on the hepatic and splenic fixed monocytes. The binding triggers the release of a proteolytic enzyme that cleaves the CR1 moiety releasing the E deficient in CR1 back to the circulation and at the same time internalizing the HP or the HP complex (with pathologic target) for destruction. As a result of the CR1 transfer reaction, CR1 numbers on the E surface are reduced.

### Characterization and Downsides of the E HP Clearance Process

Generally ≧95% of pathologic target clearance is achieved by using E HP. Since sensitized Es in the absence of the target are themselves undergoing the transfer reaction, this competitively inhibits the target clearance. The result of reduced numbers of CR1 sites on E, released back into the circulation, and with the understanding that the CR1 receptor in normal Es has a limited expression on the surface, leads to an impairment of the host immune response to other targets not targeted by the HP or other soluble immune complexes, and not related to the targeted molecules. Also, the E HP clearance process would have limitations specifically in circumstances that would require repeated rounds of treatment with HP, such as prolonged exposure to biological warfare agents or where the pathologic target is an autoimmune antibody in a chronic disease state. HP will not provide long-term protection to the host.

Furthermore, the use of mouse monoclonal antibodies on the HP manifests an immunologic reaction on the primate experimental model resulting in complement opsonization rendering these E HPs unable to clear the pathologic target from the blood via this CR1 transfer pathway due to HP damage.

In summary, problems with use of this strategy include:

Inability to retain E HP and immunity for a sufficient period (only minutes).

Transient decrease in erythrocyte CR1, which may compromise the body's natural complement opsonized clearance of pathogenic immune complexes by the E CR1 receptor and the CR1 exchange reaction.

The E HP/pathologic target is processed in a CR1 transfer reaction only in the liver (and to a lesser extent spleen) mediated by binding to the FCγR resulting in release of E with depleted CR1.

Similarly, the E HP sans pathologic target is processed in a CR1 exchange reaction only in the liver (and to a lesser extent spleen) again mediated by binding to the FCR resulting in release of E with depleted CR1, in direct competition with clearance of the E HP/target complex.

Host immune reactions to the HP decrease the efficacy of the HP to function as designed especially after multiple HP immunizations.

Usual inability to clear >99% of pathologic target.

For applications such as prophylaxis for exposure to biological weapons, and chronic long-term autoimmune disease, what is needed is a system of eliminating a pathologic target from the bloodstream that does not potentially reduce immune system efficacy. The system used should also protect and provide passive immunity to the individual for a prolonged period, and it should be capable of clearing essentially >99.9% of targets efficiently, wherever they are sequestered in the body. We anticipate that STE clearance strategies support increased target clearance over that obtainable with HP-mediated clearance.

### Redirection or Inclusion of Additional Phagocytic Compartments for the Clearance of Immune Complexes in Primates: Use of the Molecule Pair in STE I and STE II and its Characterization

An object of the present invention is to provide novel processes for the efficient and safe clearance of any pathologic target, such as an invading microorganism or an autoimmune antibody, from the bloodstream by another mechanism different from the CR1 transfer reaction.

The factor that controls the granulocyte vs. fixed monocyte clearance of the immune complex is the site of attachment of the immune complex to the E. As previously stated, attachment of the immune complex to the E CR1 site, due to its presence in discrete and limited numbers in patches on the E surface, directs the E immune complex to the monocytic macrophages fixed in the liver and spleen, where the CR1 transfer reaction occurs. However, attachment of the immune complex to any other site with homogeneous dispersion may shift the clearance to the circulating PMN granulocyte phagocytes. MP is designed to allow IC binding to those attachment sites on E different to CR1 (see Table III). All sites are immunogenic in nature, and are expressed on the E surface. In STE, the entire E MP/pathologic target complex is directed to all phagocytic compartments for clearance.

Use of the molecular pairs (MPs), for clearance from the blood of immunogen or microbe [MP (a₁a₂)], or for autoimmune antibody [MP (a-ag)], directs the attachment of the immune complex away from the CR1 site to any other surface expressed immunogenic molecules on the E and to clearance by a number of phagocytic cell compartments via phagocytosis of the E MP/target complex.

As previously stated, the MP can be attached to other non-immunogenic sites on the surface of the RBC or RBC ghost by a number of different attachment modalities. In a preferred embodiment of the present invention, E MP (a₁a₂) is an antibody pair, namely one antibody specific to the Rho (D) site on the primate or human erythrocyte covalently linked, by any method known to those skilled in the art, to another antibody specific for the pathologic target.

The following chart describes the MP on Rh positive people:

THE MP (a₁a₂) CONSTRUCT IN Rh POSITIVE PEOPLE

| | | ABILITY TO FIX |
|---|---|---|
| | FUNCTION | COMPLEMENT |
| a₁ | Attachment antibody to E at Rho (D) locus or | NO |
| | other site (other than CR1) | |
| a₂ | Capture antibody of pathologic immunogenic | YES |
| | target to be cleared | |

The attachment antibodies may be of any type or an antibody fragment (Fab)₂ or Fab devoid of the Fc region. The absence of the Fc region on the anchor antibody of all MP pairs will prevent complement fixation and activation at the MP attachment site. In this embodiment the presence of an Fc region on the attachment antibody, IgG anti Rho (D), is allowed due to its inability to fix and activate complement, known to those skilled in the art. The site of attachment for the antibody pair requires a homogeneously expressed immunogenic or other molecule on the E surface. Table III presents possible sites of attachment of the MP to the E surface. The target capture antibody must possess an intact Fc region in order to support complement fixation.

Another preferred embodiment includes an antibody-antigen pair [MP (a-ag)], wherein the attachment antibody (a) is similar to that presented in the a₁-a₂ pair, namely an anti Rho (D) antibody or antibody fragment, with differing specificities. The antibody is covalently attached to an antigen for rapid removal of the autoimmune antibody specific for the antigen circulating in the host. Again, in other embodiments the site of attachment of the a-ag pair to the E surface may be any protein, carbohydrate, or other site that is homogeneously expressed on the E surface with use of the corresponding specificity antibody excluding the CR1 site on E.

THE MP (a-ag) CONSTRUCT IN Rh POSITIVE PEOPLE

| | | ABILITY TO FIX |
|---|---|---|
| | FUNCTION | COMPLEMENT |
| a | Attachment antibody to E at Rho (D) locus or | NO |
| | other site (other than CR1) | |
| ag | Capture antigen to bind the pathologic | YES |
| | autoimmune antibody to be cleared | |

Since approximately 10-20% of people worldwide are Rho negative and do not possess the D antigen on their E cell surface, attachment antibody on [MP (a₁a₂)] and [MP (a-ag)] should be directed to a site different to the Rho (D) locus (see Table III). The following chart explains some of the preferred embodiments on Rh negative people:

| | |
|---|---|
| | ABILITY TO FIX |
| FUNCTION | COMPLEMENT |

THE MP (a₁-a₂) CONSTRUCT IN Rh NEGATIVE PEOPLE

| | | |
|---|---|---|
| a₁ | Attachment antibody fragment, devoid of Fc, at | NO |
| | any site homogeneously expressed of the E | |
| | surface other than CR1 (Rho (D) not present). | |
| a₂ | Capture antibody to immunogen to be cleared | YES |
| | from the bloodstream | |

THE MP (a-ag) CONSTRUCT IN Rh NEGATIVE PEOPLE

| | | |
|---|---|---|
| a | Attachment of antibody fragment, devoid of Fc, | NO |
| | at any site homogeneously expressed of the E | |
| | surface other than CR1 (Rho (D) not present). | |
| ag | Capture antibody of immunogen to be cleared | YES |
| | from the bloodstream | |

In preferred embodiments of the present invention:

None of the above sensitized Es, namely E [MP (a₁a₂)] and E [MP (a-ag)] are able to fix complement, by design, in the Rh positive and negative host prior to pathologic target binding.

Complement is fixed and activated post pathologic target binding only, which triggers phagocytosis.

These sensitized Es, however, themselves prior to attachment of the pathologic target, are susceptible to clearance from the bloodstream if intact Fc regions are present which will interact with the FcγRs located on all phagocytic cells. This fact would affect the E MP survival in circulation.

For longevity in blood circulation the E MP needs to be resistant to phagocytosis unless target binding and complement fixation occur. The presence of intact Fc region on the MP antibodies would drive the rapid uptake of MP sensitized E by phagocytic cells. One strategy to achieve maximal E MP survival would be to genetically engineer both target capture and MP attachment antibodies (when possessing an Fc region(s) by design) with modified Fc regions incapable of being recognized by the FcγR receptors on the fixed hepatic and splenic monocytes.

### Inhibition of the Fc Mediated Clearance of E MP Prior to Binding of their Pathologic Targets

E MPs upon proper construction may remain in the circulatory system for a maximum period of 120 days, which represents the 60-day half-life of an erythrocyte. It is known by those skilled in the art that granulocytes and fixed macrophages, including the Kupffer cells in the liver, possess surface FcγRs that attach immune complexes possessing normal Fc regions, such as E MP (Fc). It has been established that the phagocytic reaction occurs in two stages, the attachment of the Fc expressing immune complex to the Fcγ receptor, which then triggers the local pseudopod engulfing reaction. In order to phagocytize the entire E immune complex, multiple Fc determinants must be bound over the entire E surface. In preferred methods, this MP clearance sans target is blocked by any means so that the E MPs will not be prematurely cleared from the bloodstream.

It is known to those skilled in the art that methods exist to interfere with the interaction between the antibody Fc region and the FcγR. Those methods may be useful to block interaction of E MP and FcγR on phagocytic cells. One method is the use of androgens, which when delivered to phagocytic cells produce decreased FcγR1 and FcγR2 expression. Both types of receptors are expressed on all granulocytic and macrophage cells. FcγR decreased expression has no effect on immune complex (C3b) recognition by CR1 receptors on the macrophage surface and its subsequent phagocytosis. Although it is known the use of sex hormones exert a positive effect on autoimmune disorders and immune cytopenia, their use for the present invention would be restrictive.

Another method used to negate the effect of the FcγR receptors includes the introduction of excess soluble Fc to the system that would competitively inhibit the clearance reaction of the E MP with the FcγR. Lastly, as previously stated, the Fc domains responsible for complement fixation and FcγR recognition map to different loci. A recombinant Fc fragment may be constructed that will support efficient C1q binding (complement fixation), and subsequent complement activation, without being recognized by the FcγR receptor on macrophage surfaces.

In general, modification of the FcγR would prolong E MP and Eg MP survival in the host circulation. It is also the object of STE to extend the target clearance form the macrophages in the liver and spleen to include the circulating PMN phagocytes. Those skilled in the art know that the FcγR III mediates neutrophil recruitment to phagocytize immune complexes. An Fc modified region to avoid binding of the E MP or Eg MP to the FcγR on the liver and spleen macrophage may similarly preclude binding of the E MP or Eg MP to the PMNs. In this scenario, a complement trigger will support the required phagocytosis of the E MP/target/C3b and Eg MP/target/C3b complexes in vivo by the PMNs.

### E MP: Use of the Natural Phagocytic Receptors for Rapid and Efficient Target Clearance Via Phagocytosis in Multiple Phagocytic Compartments not Involving the CR1 Exchange Reaction

The present invention involves a number of embodiments that in general can be used for clearance of pathologic or other targets from the peripheral blood. These targets may be microbes, toxic chemicals, toxins, autoimmune antibody and others. Embodiments of the current invention called Selective Target Elimination (STE) fall into two categories, herein, referred to as STE I and STE II. Both support in vivo pathologic target clearance independent of the CR1 transfer reaction. STE embodiments intend to add the circulating phagocytic compartment to the liver and spleen fixed tissue monocyte phagocytic compartments, and also to exploit other natural systems in the body to achieve improved target clearance. STE embodiments are presented in parallel with HP and CR1 clearance in Table II.

Selective Target Elimination I (STE I)

STE I involves the in vivo or ex vivo sensitization of Es with the MP. This method utilizes the intact circulating red blood cells (RBC) to indirectly clear the target present in the circulation. The E is sensitized in vivo by injection of the MP into the body. Conversely, universal donor RBCs or autologous RBCs may be sensitized in vitro and the E MPs subsequently transfused into the body.

The MP in general is represented as IgG pathologic target-RBC attachment antibody fragment devoid of Fc region. The MP is composed of humanized mAbs to avoid host immune reaction against the mabs (initially of murine origin), and the target capture mAb possesses a normal Fc region suitable for complement fixation; however, this Fc region may need modification to avoid recognition by the FcγR on phagocytic cells. The circulating E MP rapidly binds any pathologic target resulting in complement fixation and activation. The E MP/target/C3b complex is cleared from the circulation in a number of phagocytic cell compartments including circulating PMNs, hepatic and splenic fixed tissue monocytes. Simultaneously, complement fixation by the E MP/target complex will also lead to immediate destruction of some microbial targets by the mechanism of complement fixation and activation of the classical complement pathway and the alternate complement pathway, known to those skilled in the art. The E MP sans target possesses no complement C3b opsonin allowing its longer term survival in the circulation.

STE I is characterized by addition of the circulating PMN phagocytic compartment for the clearance of the E/pathologic target complex along with the monocyte phagocytic compartment in the liver and spleen. Its upsides include:

Provision of a 120 day passive immunity period based on the 60 day half-life of the primate E (and can be extended by additional injection).

The inability to stimulate a host immune reaction to the immune globulin (MP) used that confers the passive immunity (antibodies used are humanized).

The potential to neutralize and clear >99.9% of a range of the pathologic targets present in the host due to the expansion of the phagocytic compartments suitable for target clearance.

The immediate neutralization and destruction of the pathologic target by complement fixation (complement trigger) prior to target clearance.

STE I may also have some downsides, namely:

Difficulties inherent to complement activation in the systemic circulation by the targets present.

Potential impairment of macrophage functions due to ingestion of intact RBCs.

Tolerance to the target might be developed.

In consideration of the potential downsides of STE I, the STE II embodiment was designed. STE II employs RBC ghosts instead of intact RBCs, thereby avoiding the phagocyte toxicity of the RBC contents. While STE IIa is independent of complement activation, STE IIb possesses a complement trigger to initiate the Eg MP/target/C3b complex phagocytic event.

### Selective Target Elimination IIa (Eg MP): Use of the Natural Apoptotic Cell Clearance Mechanism for in Vivo Clearance of Targets Present in the Circulation [Short Term Passive Immunity/See Table II]

The RBC has a life span of 120 days. As they become senescent, changes in membrane structure and integrity occur, such as phosphatidylserine (PS) exposure on the outer leaflet of the membrane; Band-3 clustering, among others. Those changes signal the RBC removal from the circulation and promote macrophage-mediated erythro-phagocytosis in the spleen and liver. This is a natural clearance mechanism occurring in the body for clearance of RBC senescent cells. It is estimated that 360 million RBCs are phagocytized every day.

Based on this natural clearance mechanism, for the STE IIa process we prepare RBC ghosts, generate the senescence markers on the ghosts and sensitize them with the MP (Eg MP). The rationale for STE IIa action is that the transfusion of Eg MP, which immediately binds the targets in vivo induces the rapid phagocytosis of both the apoptotic cell mimic with the attached MP/target complex. The Eg MP/target complex is immediately recognized as a senescent cell for clearance, in the spleen and liver, by the natural apoptotic/senescent cell clearance pathway.

Although STE I attempts to expand the phagocytic compartment to the circulating PMNs, such is not the aim of STE IIa. STE IIa uses the highly efficient apoptotic cell clearance system as a privileged mechanism for efficient in vivo target clearance just as the HP exploits the efficient CR1 exchange reaction for in vivo target clearance.

The Eg MP can be recognized and treated as a senescent apoptotic cell for clearance by the body's natural mechanism by:

Chemically modifying E of all ages by addition of phosphatidylserine (PS) on the E surface before or after MP sensitization and subsequent E lysis.

Chemically modifying E of all ages by addition of Galactose, α1,3 to human erythrocytes resulting in the creation of a senescence-associated epitope.

Lysis of E in a hypotonic solution itself should result in the surface appearance of PS and render the Eg MP an apoptotic cell mimic.

Lysis of E MP in the presence of divalent cation (Mg⁺⁺) and in the absence of ATP results in high PS exposure on the Eg MP surface, whereas other methods with ATP provide ghosts with limited surface PS expression Crosslinking of RBC surface protein such as band-3 by hetero-bifunctional cross-linking reagents or antibody cross-linking, prior to MP sensitization and subsequent lysis to produce the Eg MP.

Isolation of apoptotic RBCs by density gradient centrifugation, allowing only senescent RBCs to be sensitized and subsequently lysed to produce the necessary apoptotic mimic, Eg MP.

Any other physical/chemical treatment or other procedures resulting in the production of the apoptotic mimic or natural apoptotic Eg MP.

In STE IIa the trigger for the clearance mechanism is the transfusion of induced apoptotic mimic Eg MPs. There is no requirement for a complement trigger to initiate the apoptotic cell clearance; however, it is known that both the classical and/or the alternate pathway participate in a late stage of the clearance process. The target to be cleared is bound by the MP specific molecule pair on the Eg surface and cleared with the ghost. The binding of the target by the MP often will neutralize a toxin or the toxicity of a poisonous chemical, until the target/Eg MP can be ingested and cleared by the macrophages.

STE IIa is characterized by:

Short term passive immunity.

Inability to stimulate a host immune reaction to the immune globulin conferring the passive immunity.

The potential to clear >99.9% of the pathologic targets present in the host.

The lack of a complement trigger to initiate clearance.

An efficient and rapid rate of clearance of the pathologic target by an efficient natural mechanism

The steps of STE IIa are:

Step I: Sensitize universal donor RBCs, ABO type “O” or other autologous intact RBCs with the MP: IgG anti target-Fab anti any attachment site on the RBC other than CR1.

Step II: Treat the RBCs by a physical or chemical process that will induce the sensitized RBC to become recognized as apoptotic. This may include lysis of the intact E MP to produce Eg MP or any physical or chemical treatment known to those skilled in the art that will induce the apoptotic cell clearance mechanism by recognition of PS on the Eg MP surface. It is known that lysis of intact RBCs in the presence of divalent cations (Mg⁺⁺) results in the high level of expression of PS on the RBC ghost surface. It is also known that the level can be reduced by the concomitant addition of ATP to the lysis process which would allow the translocase enzyme to actively bury the surface PS between the membrane layers, thus offering a surface PS modulation mechanism. It is known to those skilled in the art that apoptotic RBCs are phagocytized in a natural mechanism by the monocyte phagocytic compartments.

Step III: The target-specific MP sensitized apoptotic mimic RBCs (Eg MPs) are transfused into the host, whereupon, the targets immediately bind to the Eg MPs. This is supported by studies in the E HP system, indicating rapid binding of the targets in a few minute period to the E HPs upon HP injection.

Step IV: The mimic apoptotic state of the Eg induces efficient macrophage phagocytosis of the Eg MP by the natural clearance mechanism.

### Kinetics of Eg MP Clearance of a Pathologic Target by STE IIa

The Eg MP in this embodiment will possess a large number of PS sites on the ghost surface.

The transfused Eg MP will immediately bind the pathologic target if present in the circulation

The exposed PS will be bound to the PS receptor on the fixed tissue monocytes on the spleen and liver, where they will be immediately cleared due to their recognition as scenescent apoptotic cells.

The duration of Eg MP in the circulation in this embodiment is limited to a period of hours.

No complement fixation is necessary to trigger phagocytosis by this natural apoptotic cell clearance pathway, however, PS exposed on the ghost erythrocyte surface has been shown to activate the alternate complement pathway and result in deposition of C3b onto the Eg MP. This may explain the rapid nature of the apoptotic cell clearance pathway.

### Selective Target Elimination IIb (Eg MP): Long Term Passive Immunity

In STE IIa the high Eg surface expressing PS level functions to preprogram the Eg MP for immediate clearance by the apoptotic cell clearance pathway, and the period of immunity is short-lived. To lengthen the period of passive immunity to possibly months the STE IIb method was designed.

Herein, the Eg is prepared under experimental conditions resulting in low or no PS surface exposure. PS is neutralized or effectively “buried” by any mechanism known to those skilled in the art, including binding of annexin V IgG anti PS, or MP (IgG anti pathologic target-Fab anti PS), or any other mechanism, which effectively blocks the Eg surface PS from recognition by the macrophage PS surface receptor.

The Eg is next sensitized with the MP specific for the target to be cleared. Since it is known that PS is recognized by the PS receptor on the macrophage surface and provides the initial site of phagocyte attachment to the Eg MP, burying the PS would support prolonged survival of the Eg MP in the circulation, whereupon the targets marked for clearance are bound forming the Eg MP/target complex. Upon complex formation, complement is fixed and the Eg MP/target/C3b complex is phagocytized by the macrophages through the CR1 scavenger receptor on the macrophage surface. Herein, the C3b will be the sole signal to induce target complex phagocytosis. The antibodies of the MP will be humanized and may possess a modified Fc region to avoid recognition by the FcγR on the macrophages in the liver and spleen, adding to the in vivo survival of Eg MP.

STE IIb is characterized by:

A possible increase in the number of phagocytic compartments.

Long term passive immunity.

Inability to stimulate a host immune reaction to the immune globulin conferring the passive immunity.

The potential to clear >99.9% of the targets present in the host.

The presence of a complement trigger.

The rapid and continuous clearance of the specific target by a natural phagocytic compartment.

The steps of STE IIb are:

Step I: Sensitize intact universal donor RBCs, ABO type “O” or autologous intact RBCs with the MP:IgG anti target-Fab anti any attachment site on the RBC other than CR1.

Step II: Lyse the E MP by any method resulting in low surface exposure of PS on the Eg MP surface. Since the object of this embodiment is to prolong survival of the Eg MP in the circulation, the PS sites present on the Eg MP surface can be neutralized as described above. In one embodiment, binding an additional MP to the Eg MP, namely IgG anti target-Fab anti PS will prevent macrophage recognition of the apoptotic cell mimic, the Eg MP.

Step III: Bind the target for clearance to the Eg MP thereby activating the complement trigger by the opsonization of C3b to the Eg MP surface. This C3b will be the only signal to induce Eg MP phagocytosis by the natural mechanism in fixed monocytes in the liver and spleen. The antibodies of the MPs used herein will be humanized and possess a modified Fc region not recognized by the Fcγ receptor in macrophages, adding to the in vivo survival of the Eg MP.

Step IV: Clearance of the Eg MP/target/C3b opsonized complex by the macrophages in the liver and spleen.

### Kinetics of Eg MP Clearance of a Pathologic Target by STE IIb

The Eg MP will possess a small number of (or no) PS sites on the ghost surface. The few PS sites present will be “buried” by complexation with the MP (IgG anti target-IgG anti PS) preventing macrophage recognition of the Eg MP and its prolonged survival in the circulation.

The transfused Eg MPs will immediately bind the pathologic target if present in the circulation.

The inability of the Eg MP itself to trigger phagocytosis due to blocking of surface PS sites and modification of the Fc regions on the antibody present will support the extended Eg MP survival in the circulation.

Complexation of the target with the Eg MP will subsequently result in complement fixation and the opsonization of the Eg MP/target complex with C3b.

The C3b generated by a complement trigger will mark the Eg MP/target complex for clearance by the fixed monocytes of the liver and spleen mediated by their surface C3b receptors.

The clearance of the target will similarly continue in the body as long as the Eg MP exists in the circulatory system.

### STE IIc (Eg MP): Another Embodiment for Use of the Natural Apoptotic Cell Clearance Mechanism for Prolonged in Vivo Clearance of Targets Present in the Circulation

STE IIc embodiment combines the characteristics of STE IIa and IIb. RBC ghosts are prepared under experimental conditions to promote aggregates of the band-3 polypeptide, a major RBC membrane protein. It is well known by those skilled in the art that aggregation of band-3 generates neo-antigens recognized by natural auto-antibodies present in the host circulation. Furthermore phagocytosis of damaged RBCs, by the macrophages in the liver and spleen, is mediated by the antibody binding to clustered band-3 antigen and activation of the alternative complement pathway.

It is also known by those skilled in the art that RBC infected with Plasmodium (iRBC), parasitic agent of Malaria disease, present membrane alterations such as clustering of the band-3 protein promoting the RBC clearance through the phagocytic compartments. Moreover, it was shown in vitro that anti-malarial drugs considerably reduced the binding of the auto-antibodies to the band-3 of the iRBC by an unknown mechanism, resulting in the failure of iRBC phagocytosis, Shalmiev et al., Trans R Soc of Trol Med Hyg, Vol.90, pages 558-562 (1996).

Although anti-malaria drugs produce some minor side effects, they are recommended as prophylaxis for travelers to Malaria endemic areas. From a practical standpoint to secure a strong response against any pathological target there would not be any restriction for use of this type of pharmacologic substance.

In STE IIc embodiment the use of MP sensitized RBC ghosts characterized by clustering of band-3 and low to no PS surface exposure, co-administered with anti-malaria drugs, may promote in vivo survival of the Eg MP. The clearance signal for the Eg MP is provided by the band-3 crosslinking after blood levels of the drug have been allowed to diminish.

STE IIc embodiment is then characterized by:ng term passive immunity

Inability to stimulate a host immune reaction to the immune globulin conferring the passive immunity

The potential to clear >99.9% of a range of targets in the host

Rapid and continuous neutralization of the specific targets

### Kinetics of Eg MP clearance of a Pathologic Target by STE IIc

The Eg MP will possess no PS exposure on the ghost membrane surface. The Eg possesses band-3 proteins that are clustered, which is a marker for senescent and apoptotic red blood cells that triggers the clearance of this cell population. Band-3 clustering may be accomplished by use of hetero-bifunctional linkers. Since it is known by those skilled in the art that anti-malaria drugs such as chloroquine blocks the in vitro, phagocytosis of antibody opsonized malaria containing E and that drug removal will support the phagocytic event, STE IIc was configured to exploit this effect.

Aggregation of band-3 in MP sensitized Egs and co-administration of chloroquine will support the lengthened survival of the Eg MP in the circulation. Since the chloroquine functions to inhibit antibody opsonized clearance of the red blood cell, the Eg MP and the Eg MP/target complex are cleared only after the levels of chloroquine drop appreciable as a result of a discontinuation of chloroquine administration. This decrease of in vivo chloroquine levels is the trigger necessary for clearance of the MP sensitized Egs in the presence or absence of the target.

A method for blood-bome pathogen clearance in a patient in vivo is provided comprising (a) preparing at least one erythrocyte ghost having senescence markers; (b) sensitizing at least one of the erythrocyte ghosts with at least one molecule pair ex vivo to form a sensitized erythrocyte ghost molecule pair; (c) administering an effective amount of the sensitized erythrocyte ghost molecule pair to a patient; and (d) effecting the binding of the sensitized erythrocyte ghost molecule pair to a specific pathological agent present in the patient's blood resulting in an erythrocyte ghost molecule pair pathological agent, and clearing the erythrocyte ghost molecule pair pathological agent from the patient's blood.

A method for forming a sensitized erythrocyte is provided comprising (a) obtaining at least one erythrocyte; (b) biotinylating the erythrocyte to form a biotinylated erythrocyte; (c) obtaining at least one monoclonal antibody specific to a target; (d) biotinylating the monoclonal antibody to form a biotinylated monoclonal antibody; (e) binding the biotinylated erythrocyte to avidin; and (f) binding the avidin having the biotinylated erythrocyte to the biotinylated monoclonal antibody to form a sensitized erythrocyte.

A method for forming a sensitized erythrocyte is provided comprising (a) obtaining at least one erythrocyte; (b) biotinylating the erythrocyte to form a biotinylated erythrocyte; (c) obtaining at least one monoclonal antibody specific to a target; (d) biotinylating the monoclonal antibody to form a biotinylated monoclonal antibody; (e) binding the biotinylated erythrocyte to streptavidin; and (f) binding the streptavidin having the biotinylated erythrocyte to the biotinylated monoclonal antibody to form a sensitized erythrocyte.

A method for forming a sensitized erythrocyte is provided comprising (a) obtaining at least one erythrocyte; (b) selecting a high-affinity binding pair; (c) treating the erythrocyte with a first member of said high-affinity binding pair; (d) obtaining at least one monoclonal antibody specific to a target; (e) treating the monoclonal antibody with a second member of the high-affinity binding pair; and (f) combining the treated erythrocyte with the treated monoclonal antibody to form a sensitized erythrocyte. This method includes wherein the first member of the highaffinity binding pair is N-hydroxysuccinimide ester, biotin, or biotinphosphatidylethanolamine; and wherein the second member of the high-affinity binding pair is avidin or streptavidin.

A composition is provided comprising an erythrocyte and a molecule pair antibody wherein the molecule pair antibody is bound to the erythrocyte at the Rho (D) locus of the erythrocyte, and wherein the molecule pair antibody comprises IgG anti Rho (D) covalently bound to a monoclonal antibody specific for a target, and wherein the IgG anti Rho (D) has an Fc region.

A method for prolonging the ability to eliminate pathological agents from the blood of a patient is provided comprising administering to a patient at least one sensitized erythrocyte ghost having a molecule pair antibody complex that is capable of binding a pathological agent, including wherein the sensitized erythrocyte ghost includes a band 3 surface polypeptide, and including wherein the sensitized erythrocyte ghost exhibits no surface appearance of phosphatidylserine; and administering an effective amount of an anti-malaria drug to the patient to prevent elimination of the sensitized erythrocyte ghost molecule pair antibody for prolonging the patient's ability to eliminate the pathological agent.

A method for elimination of pathological agents from the blood of a patient is provided comprising administering to the patient at least one sensitized erythrocyte having a molecule pair antibody that is capable of binding a pathological agent at a site other than the CR1 receptor of the sensitized erythrocyte and eliminating the pathological agent from the patient's blood, and including adding an effective amount of soluble Fc that is effective for inhibiting the clearance reaction of the sensitized erythrocyte molecule pair.

A method for blood-borne pathogen clearance in a patient in vivo is provided comprising administering to a patient an effective amount of a molecule pair, wherein the molecule pair is prepared using humanized or non-humanized antibodies, allowing the molecule pair to bind to a specific site on at least one erythrocyte surface different from CR1 thereby forming a sensitized erythrocyte molecule pair, and allowing the sensitized erythrocyte molecule pair to bind to a specific pathological target in the patient's blood to any site on the erythrocyte other than the CR1 resulting in an erythrocyte molecule pair pathological target, and clearing the erythrocyte molecule pair pathological target from the patient's blood.

A method for blood-borne pathogen clearance in a patient in vivo is provided comprising administering to a patient an effective amount of a molecule pair, wherein the molecule pair is prepared using humanized or non-humanized antibodies, allowing the molecule pair to bind to a specific site on at least one erythrocyte ghost surface thereby forming a sensitized erythrocyte ghost molecule pair, and allowing the sensitized erythrocyte ghost molecule pair to bind to a specific pathological target in the patient's blood to any site on the erythrocyte resulting in an erythrocyte ghost molecule pair pathological target, and clearing the erythrocyte ghost molecule pair pathological target from the patient's blood.

A method for elimination of pathological agents from the blood of a patient is provided comprising administering to the patient at least one sensitized erythrocyte having a molecule pair antibody that is capable of binding a pathological agent at a site other than the CR1 receptor, including wherein the molecule pair antibody comprises two antibodies that are covalently linked, wherein one of the antibodies is specific for binding to an erythrocyte receptor site and the other antibody is specific to the pathological agent, and including wherein the antibody specific to the pathological agent possesses an intact Fc region, and eliminating the pathological agent from the patient's blood independent of the CR1 exchange reaction.

A method for elimination of pathological agents from the blood of a patient is provided comprising administering to the patient at least one sensitized erythrocyte having a molecule pair antibody that is capable of binding a pathological agent at a site other than the CR1 receptor, eliminating the pathological agent from the patient's blood independent of the CR1 exchange reaction, and repeating the above steps as desired for extending the ability to eliminate pathological agents from the blood of the patient.

A method for blood-borne pathogen clearance in a patient in vivo is provided comprising preparing at least one erythrocyte ghost having senescence markers, sensitizing at least one of the erythrocyte ghosts with at least one molecule pair ex vivo, administering an effective amount of the sensitized erythrocyte ghost molecule pair to a patient, and allowing the sensitized erythrocyte ghost molecule pair to bind to a specific pathological agent present in the patient's blood resulting in an erythrocyte ghost molecule pair pathological agent, and clearing the erythrocyte ghost molecule pair pathological agent from the patient's body.

TABLE I

| | IN VIVO | | CLEAR- | | | DURATION OF |
|---|---|---|---|---|---|---|
| | CLEAR- | | ANCE | SITE OF | RATE OF | FUNCTION OF |
| | ANCE | OPSO- | MEDIATED | CLEAR- | CLEAR- | PROTECTIVE |
| | OF | NIZATION | BY | ANCE | ANCE | SYSTEM |
| A | Immune | w/o C3b | Phagocytic | Primarily | Slow | Lifetime |
| | complex | IgG only | cells | monocyte | | |
| | (IC) | | | spleen and | | |
| | | | | liver, | | |
| | | | | possibly | | |
| | | | | PMN in | | |
| | | | | circulation | | |
| B | Immune | IgG, C3b | Phagocytic | Primarily | More rapid | Lifetime |
| | complex | | cells | monocyte | | |
| | | | | spleen and | | |
| | | | | liver, | | |
| | | | | possibly | | |
| | | | | PMN in | | |
| | | | | circulation | | |
| C | Immune | IgG, C3b | E CR1 | Exclusively | Very rapid | Lifetime |
| | complex | | (CR1 transfer | monocytes | | |
| | | | reaction) | spleen and | | |
| | | | | liver | | |
| D | Target | IgG only | E HP | Exclusively | Very rapid | Minutes to |
| | (IC) | (C3b not | (CR1 transfer | monocytes | | hours |
| | | required) | reaction) | spleen and | | |
| | | | | liver | | |
| E | Target | IgG, C3b | E MP | Primarily | More rapid | 120 Days |
| | (IC) | | Phagocytosis | monocytes | | |
| | | | STE I | spleen and | | |
| | | | | liver | | |
| | | | | possibly | | |
| | | | | PMN in | | |
| | | | | circulation | | |
| F | Target | IgG only, | Eg MP | Spleen and | Very rapid | Minutes to |
| | (IC) | (C3b not | Phagocytosis | liver | | hours |
| | | required) | STE IIa | monocytes | | |
| G | Target | IgG, C3b | Eg MP | Spleen and | More rapid | Days |
| | (IC) | | Phagocytosis | liver | | (long-lived) |
| | | | STE IIb | monocytes | | |
| H | Target | Complement | Eg MP | Spleen and | More rapid | Days |
| | (IC) | (Alternate | Phagocytosis | liver | | (long-lived) |
| | | Pathway) | STE IIc | monocytes | | |

TABLE II

| Process | | | | | |
|---|---|---|---|---|---|
| Characterization | STE I | STE IIa | STE IIb | STE IIc | HP CR1 |
| Globulin type | Antibody Pair | Antibody Pair | Antibody Pair | Antibody Pair | Antibody |
| (RBC | MP (molecule | MP (molecule | MP (molecule | MP (molecule | Pair |
| attachment and | pair) | pair) | pair) | pair) | HP (heteropolymer) |
| target capture | | | | | |
| antibodies) | | | | | |
| RBC | All sites other | All sites and | All sites and | All sites and | CR1 only |
| Attachment | than CR1 and | artificial sites | artificial sites | artificial sites | |
| site | artificial sites | | | | |
| Attachment | Fab, (Fab)₂, | Fab, (Fab)₂, and | Fab, (Fab)₂, | Fab, (Fab)₂, IgG | IgG anti CR1 |
| antibody | (devoid of Fc) | IgG with normal | (devoid of Fc) | chloroquine | only (normal |
| | incapable of | Fc(fix | incapable of | negates | Fc) |
| | fixing | complement) | fixing | presence of | |
| | complement | | complement | normal Fc | |
| | or complete | | | | |
| | IgG anti D | | | | |
| Pathologic | IgG (must | Fab, (Fab)₂, IgG | Fab, (Fab)₂, | Fab, (Fab)₂, IgG | Fab, (Fab)₂, |
| target capture | possess Fc) or | (normal Fc) | IgG | chloroquine | IgG |
| antibody | modified Fc | | (normal Fc) | negates | (normal Fc) |
| | to fix | | | presence of | |
| | complement | | | normal Fc | |
| Globulin | Injection of | Ex vivo RBC | Ex vivo RBC | Ex vivo RBC | Injection of |
| delivery method | MP or | ghost (high | ghost (low | ghost (low | HP |
| | transfusion of | surface PS) | surface PS) | surface PS) | |
| | E MP | sensitization of | sensitization of | sensitization of | |
| | | RBCs type O or | type O or | RBCs type O or | |
| | | autologous and | autologous, | autologous, and | |
| | | their transfusion | and their | their transfusion | |
| | | | transfusion | | |
| Duration of | Long-lasting | Intermediate | Long-lasting | Long-lasting | Short-lived |
| passive | | lasting | | | |
| immunity | | | | | |
| period | | | | | |
| Other antibody | Humanized | Humanized | Humanized | Humanized | None |
| requirements | | | | | |
| attachment | | | | | |
| antibody | | | | | |
| Target capture | Humanized | Humanized | Humanized | Humanized | None |
| antibody | modification | modification | modification of | antibody | |
| | of Fc to avoid | only | Fc to avoid | fragment (no | |
| | FcγR | | FcγR | Fc required) | |
| Ability to | Yes | Yes | Yes | Yes | No |
| neutralize and | | | | | |
| inactivate | | | | | |
| microbial target | | | | | |
| upon capture by | | | | | |
| complement | | | | | |
| fixation and | | | | | |
| activation | | | | | |
| Phagocytic | Multiple | Multiple | Multiple | Multiple | Single |
| compartments | 1. Liver (fixed | 1. Spleen (fixed | 1. Spleen (fixed | 1. Spleen (fixed | 1. Liver (fixed |
| utilized | monocytes | monocytes) | monocytes) | monocytes) | monocyte) |
| | 2. Spleen | 2. Liver (fixed | 2. Liver (fixed | 2. Liver (fixed | 2. Spleen (fixed |
| | (fixed | monocytes | monocytes | monocytes | monocytes) |
| | monocytes) | | 3. Possibly | 3. Possibly | |
| | 3. Possibly | | circulating | circulating | |
| | circulating | | PMN | PMN | |
| | PMN | | | | |
| Event triggering | Complement | Transfusion of | Complement | Cessation of | Injection |
| clearance | fixation of | apoptotic cell | fixation of Eg | chloroquine | |
| | target MP | mimic E MP | MP | administration | |
| | RBC complex | ghosts | | | |
| Ability to | Yes | No | Yes | Yes | No |
| extend passive | | | | | |
| immunity | | | | | |
| period | | | | | |
| Process compromises | Not | Not | Not | Not | Yes, RBCs |
| host | anticipated | anticipated | anticipated | anticipated | loose CR1 |
| immune system | | | | | receptor |
| Capability of | Theoretical | Theoretical | Theoretical | Theoretical | Data indicates |
| clearance of | | | | | ≧95% |
| >99.9% of | | | | | clearance |
| pathologic | | | | | |
| targets | | | | | |
| Host Range | Human and | Human and | Human and all | Human and | Human and |
| | all animal | All animal | animal | All animal | animal |
| | | | | | primates only |

TABLE III
SURFACE RECEPTORS EXPRESSED IN ALL THE PHAGOCYTIC CELL
COMPARTMENTS AND THEIR GRANULAR CONTENT

| | | | | | | IC | | | | |
|---|---|---|---|---|---|---|---|---|---|---|
| | | | IC | | | Adherence | IC | | | |
| | | | Phago- | Chemo- | Chemo- | Phago- | Adherence | Enzyme Content of Granules | | |
| | | Receptor | cytosis | attractant | attractant | cytosis | Phago- | | acid | alkaline |
| | | for IgE | Receptor | Receptor | Receptor | Receptor | cytosis | peroxi- | phospha- | phospha- |
| | | FCgR | FCγR | C3aR | C5aR | CR1 | CR₃ | dase | tase | tase |
| GRANULOCYTE | NEUTROPHIL | — | + | + | + | + | + | + | + | + |
| | EOSINOPHIL | LOW | + | +? | + | + | + | + | + | |
| | | AFFINITY | | | | | | | | |
| | BASOPHIL | HIGH | + | + | + | + | + | + | | |
| | | AFFINITY | | | | | | | | |
| | MAST CELL | HIGH | + | + | + | + | | | + | + |
| | | AFFINITY | | | | | | | | |
| MONOCYTE | CIRCULATING | + | + | | | + | + | | | |
| | BLOOD | | | | | | | | | |
| | MONOCYTE | | | | | | | | | |
| | KUPFFER | | + | | | + | | | | |
| | CELLS IN LIVER | | | | | | | | | |
| | INTRAGLO- | | + | | | + | | | | |
| | MERULAR | | | | | | | | | |
| | MESANGIUM | | | | | | | | | |
| | OF THE KIDNEY | | | | | | | | | |
| | ALVEOLAR | | + | | | + | | | | |
| | MACROPHAGES | | | | | | | | | |
| | IN THE LUNG | | | | | | | | | |
| | SEROSAL | | + | | | + | | | | |
| | MACROPHAGES | | | | | | | | | |
| | BRAIN | | + | | | + | | | | |
| | MICROGLIA | | | | | | | | | |
| | SPLEEN SINUS | | + | | | + | | | | |
| | MACROPHAGES | | | | | | | | | |
| | LYMPH NODE | | + | | | + | | | | |
| | SINUS | | | | | | | | | |
| | MACROPHAGES | | | | | | | | | |

TABLE IV
SITES FOR POSSIBLE ATTACHMENT
OF MP TO THE E SURFACE

17-Beta-Estradiol Receptor
Anion Exchange Protein (AE1)
Aquaporin 1 Channel Protein
Band-3
Blood Group Antigens
Cell Age Specific Surface Protein (part lost in senescent cells)
Ceruloplasm Receptor
Chemokine Receptors
Concanavalin A Receptors
CR1 (Knops System Antigens)
DAF (Cromer System Antigens)
Folate Binding Protein (FBP) Receptors
Glycophorin A Receptor
Hyaluronan Receptor
Integrin Receptor
Interleukin 2 Receptors
Laminin Receptor
Lectin Receptor
Lymphocyte Associated Antigen 3
MIC-2 Protein
MSP-1 Peptide Receptor
Neurothelin
Platelet Glycoprotein IV
Tamm-Horsfall Glycoprotein Receptors
Transferrin Receptor
And Any Other Surface Protein, Carbohydrate, and artificial site

TABLE V

| | |
|---|---|
| Ab or a | Any immunoglobulin type (IgG, IgM, IgA, IgE, etc.) or |
| | antibody fragment such as (Fab)₂ or Fab |
| Ag or ag | Any immunogenic molecule with specificity for any |
| | pathologic antibody, often an autoimmune antibody |
| E HP | Sensitization of the erythrocyte with the antibody pair that |
| | binds to the CR1 site on the erythrocyte surface exclusively. |
| E HP (antigen) | Sensitization of the erythrocyte with the antibody and |
| | antigen pair that binds to the CR1 site on the erythrocyte |
| | surface exclusively. |
| E HP Target | Clearance complex to remove the pathologic target from the |
| pathologic target | blood. The target may be microbial or any that is |
| (virus or cell with | immunogenic and determines the specificity of the capture |
| surface antigens) | antibody. |
| E MP (a₁a₂) | Sensitization of the erythrocyte with the antibody pair that |
| | binds to any site other than CR1 on the erythrocyte surface. |
| E MP (a₁-a₂) Target | Clearance complex to remove the pathologic target from the |
| pathologic target | blood. The target may be microbial or any that is |
| (virus or cell with | immunogenic and determines the specificity of the capture |
| surface antigens) | antibody. |
| E MP (a-ag) | Sensitization of the erythrocyte with the antibody and |
| | antigen pair that binds to any site other than CR1 on the |
| | erythrocyte surface. |
| E MP(a-ag)Target | Clearance complex to remove the pathologic target from the |
| pathologic target | blood. The target is antibody in nature due to the |
| (autoimmune | requirement for binding to the capture antigen and must have |
| antibody specific | specificity for the antigen. |
| for the Ag on the | |
| sensitized E) | |
| HP | Heteropolymer/two antibody molecules covalently joined |
| | where one has specificity for CR1 and the other has specificity |
| | for a pathologic target. |
| HP (antigen) | Heteropolymer/one antibody molecules covalently attached |
| | to an antigen where the antibody has CR1 specificity and the |
| | antigen is reactive with some pathologic or autoimmune antibody. |
| IC | Immune complex, antigen and antibody complex, also |
| | antibody fragment and antigen complex. |
| IC (C3b) | Immune complex where antibody possesses an FC region and |
| FC fragment present | fixes and activates complement resulting in deposition of |
| C3b present | C3b. |
| IC (IgG) | Immune complex where antibody possesses an FC region. |
| FC fragment present | |
| MP | Molecule pair, consisting of two types MP a₁a₂ and MP a-ag |
| MP (a₁a₂) | Molecule pair, consisting of two antibodies or antibody |
| | fragments with different specificities covalently attached in |
| | any manner that does not compromise the specific |
| | interaction between the two antibody or fragment |
| | interactions with their immunogenic targets. One antibody |
| | or fragment is specific to a surface protein on the |
| | erythrocyte, excluding the CR1 site, and another antibody or |
| | fragment that is specific to an expressed immunogen on the |
| | surface of the pathologic microbial or other target to be |
| | cleared from the circulatory system. |
| MP (a-ag) | Molecule pair, consisting of one antibody and one antigen |
| | where the antibody or fragment is specific to a surface |
| | protein on the erythrocyte, excluding the CR1 site, and it is |
| | covalently coupled to an antigen that is specific to a |
| | pathologic antibody usually autoimmune antibody, without |
| | disruption of either function. |

Whereas particular embodiments of this invention have been described for purposes of illustration, it will be evident to those persons skilled in the art that numerous variations of the details of the present invention may be made without departing from the invention as defined in the appended claims.

We claim:

1. A method for blood-borne pathogen clearance in a patient in vivo comprising:
(a) preparing at least one erythrocyte ghost having senescence markers;
(b) sensitizing at least one of said erythrocyte ghosts with at least one molecule pair ex vivo to form a sensitized erythrocyte ghost molecule pair;
(c) administering an effective amount of said sensitized erythrocyte ghost molecule pair to a patient; and
(d) effecting the binding of said sensitized erythrocyte ghost molecule pair to a specific pathological agent present in said patient's blood resulting in an erythrocyte ghost molecule pair pathological agent, and clearing said erythrocyte ghost molecule pair pathological agent from said patient's blood.

2. A method for forming a sensitized erythrocyte comprising:
(a) obtaining at least one erythrocyte;
(b) biotinylating said erythrocyte to form a biotinylated erythrocyte;
(c) obtaining at least one monoclonal antibody specific to a target;
(d) biotinylating said monoclonal antibody to form a biotinylated monoclonal antibody;
(e) binding said biotinylated erythrocyte to avidin; and
(f) binding said avidin having said biotinylated erythrocyte to said biotinylated monoclonal antibody to form a sensitized erythrocyte.

3. A method for forming a sensitized erythrocyte comprising:
(a) obtaining at least one erythrocyte;
(b) biotinylating said erythrocyte to form a biotinylated erythrocyte;
(c) obtaining at least one monoclonal antibody specific to a target;
(d) biotinylating said monoclonal antibody to form a biotinylated monoclonal antibody;
(e) binding said biotinylated erythrocyte to streptavidin; and
(f) binding said streptavidin having said biotinylated erythrocyte to said biotinylated monoclonal antibody to form a sensitized erythrocyte.

4. A method for forming a sensitized erythrocyte comprising:
(a) obtaining at least one erythrocyte;
(b) selecting a high-affinity binding pair;
(c) treating said erythrocyte with a first member of said high-affinity binding pair;
(d) obtaining at least one monoclonal antibody specific to a target;
(e) treating said monoclonal antibody with a second member of said high-affinity binding pair; and
(f) combining said treated erythrocyte with said treated monoclonal antibody to form a sensitized erythrocyte.

5. The method of claim 4 including wherein:
(a) said first member of said high-affinity binding pair is N-hydroxysuccinimide ester, biotin, or biotin-phosphatidylethanolamine; and wherein
(b) said second member of said high-affinity binding pair is avidin or streptavidin.

6. A composition comprising an erythrocyte and a molecule pair antibody wherein said molecule pair antibody is bound to said erythrocyte at the Rho (D) locus of said erythrocyte, and wherein said molecule pair antibody comprises IgG anti Rho (D) covalently bound to a monoclonal antibody specific for a target, and wherein said IgG anti Rho (D) has an Fc region.

7. A method for prolonging the ability to eliminate pathological agents from the blood of a patient comprising:
(a) administering to a patient at least one sensitized erythrocyte ghost having a molecule pair antibody complex that is capable of binding a pathological agent;
(b) including wherein said sensitized erythrocyte ghost includes a band 3 surface polypeptide, and including wherein said sensitized erythrocyte ghost exhibits no surface appearance of phosphatidylserine; and
(c) administering an effective amount of an anti-malaria drug to said patient to prevent elimination of said sensitized erythrocyte ghost molecule pair antibody for prolonging the ability to eliminate said pathological agent.

8. A method for elimination of pathological agents from the blood of a patient comprising: administering to said patient at least one sensitized erythrocyte having a molecule pair antibody that is capable of binding a pathological agent at a site other than the CR1 receptor of said sensitized erythrocyte and eliminating said pathological agent from said patient's blood, and including adding an effective amount of soluble Fc that is effective for inhibiting the clearance reaction of said sensitized erythrocyte molecule pair.

9. A method for blood-borne pathogen clearance in a patient in vivo comprising:
(a) administering to a patient an effective amount of a molecule pair, wherein said molecule pair is prepared using humanized or non-humanized antibodies;
(b) allowing said molecule pair to bind to a specific site on at least one erythrocyte surface different from CR1 thereby forming a sensitized erythrocyte molecule pair; and
(c) allowing said sensitized erythrocyte molecule pair to bind to a specific pathological target in said patient's blood to any site on said erythrocyte other than the CR1 resulting in an erythrocyte-molecule pair-pathological target, and clearing said erythrocyte-molecule pair-pathological target from said patient's blood.

10. A method for blood-borne pathogen clearance in a patient in vivo comprising:
(a) administering to a patient an effective amount of a molecule pair, wherein said molecule pair is prepared using humanized or non-humanized antibodies;
(b) allowing said molecule pair to bind to a specific site on at least one erythrocyte ghost surface thereby forming a sensitized erythrocyte ghost molecule pair; and
(c) allowing said sensitized erythrocyte ghost molecule pair to bind to a specific pathological target in said patient's blood to any site on said erythrocyte resulting in an erythrocyte ghost molecule pair pathological target, and clearing said erythrocyte ghost molecule pair pathological target from said patient's blood.

11. A method for elimination of pathological agents from the blood of a patient comprising:
(a) administering to said patient at least one sensitized erythrocyte having a molecule pair antibody that is capable of binding a pathological agent at a site other than the CR1 receptor, including wherein said molecule pair antibody comprises two antibodies that are covalently linked, wherein one of said antibodies is specific for binding to an erythrocyte receptor site and the other antibody is specific to said pathological agent, and including wherein said antibody specific to said pathological agent possesses an intact Fc region; and
(b) eliminating said pathological agent from said patient's blood independent of the CR1 exchange reaction.

12. A method for elimination of pathological agents from the blood of a patient comprising:
(a) administering to said patient at least one sensitized erythrocyte having a molecule pair antibody that is capable of binding a pathological agent at a site other than the CR1 receptor;
(b) eliminating said pathological agent from said patient's blood independent of the CR1 exchange reaction; and
(c) repeating steps (a) and (b) for extending the ability to eliminate pathological agents from the blood of said patient.

13. A method for blood-borne pathogen clearance in a patient in vivo comprising:
(a) preparing at least one erythrocyte ghost having senescence markers;
(b) sensitizing at least one of said erythrocyte ghosts with at least one molecule pair ex vivo;
(c) administering an effective amount of said sensitized erythrocyte ghost molecule pair to a patient; and
(d) allowing said sensitized erythrocyte ghost molecule pair to bind to a specific pathological agent present in said patient's blood resulting in an erythrocyte ghost molecule pair pathological agent, and clearing said erythrocyte ghost-molecule pair-pathological agent from said patient's body.
